(19) United States
(12) Patent Application Publication
(10) Pub. No.: US 2007/0275365 A1
(43) Pub. Date: Nov. 29, 2007

(54) Human Corneal Endothelial Cells and Methods of Obtaining and Culturing Cells for Corneal Cell Transplantation

(76) Inventor: Ge Ming Lui, Honolulu, HI (US)

Correspondence Address: JACOBSON HOLMAN PLLC, 400 SEVENTH STREET N.W., SUITE 600, WASHINGTON, DC, 20004, US

(21) Appl. No.: 10/575,245

(22) PCT Filed: Oct. 7, 2004
(86) PCT No.: PCT/US04/32933

§ 371 Date: Apr. 13, 2007

Related U.S. Application Data
(60) Provisional application No. 60/510,344, filed on Oct. 10, 2003.

(51) Int. Cl. C12N 5/08; A61K 35/44

(52) U.S. Cl. 435/1.3; 435/1.1; 435/289.1; 435/304.1; 435/305.1; 435/371; 435/374; 435/378; 435/405; 435/408

(57) ABSTRACT

A method for isolation of human corneal endothelial cells by non-enzymatic harvesting and culturing of endothelial cells in vitro from critical density in a system consisting of extracellular matrix protein coated plastic dishes and growth factor enriched culture medium are used to generate expanded sub-populations of endothelial cells for cell replacement procedures or transplantation in denuded donor corneas. Special procedures are included in the process for the removal of the native corneal endothelium from the donor corneas and the seeding of the culture corneal endothelial cells onto the denuded corneas for transplantation purposes.

This patent application claims priority to U.S. patent application Ser. No. 60/510,344 filed Oct. 10, 2003, and is incorporated by reference herein as if set forth in its entirety.

### BACKGROUND OF THE INVENTION

1. Field of Invention

This patent describes improved methods of dissecting, seeding and subsequent propagation of pure culture of human corneal endothelial cells on extracellular matrix.

2. Description of Prior Art

For various reasons, the corneal portions of eyes may need to be surgically repaired or replaced. For example, the cornea may become scratched or scarred or otherwise physically damaged, greatly hindering sight. The cornea is also subject to the effects of various degenerative diseases, mandating replacement if the patient is to have normal or even near normal vision.

The cornea of the human eye is a specialized structure made up of substantially parallel relatively compacted layers of tissue. The outermost or most superficial layer of the cornea is the epithelial layer. This is a protective layer of tissue which regenerates if injured. Moving inwardly in the eye is the base surface of the epithelial layer known as Bowman's membrane. Immediately adjacent the Bowman's membrane is the stroma of the cornea, which is an extra-cellular collagen architectural matrix with scattered keratocytic cells. The stroma layer is bounded at its deepest level by a cuticular, a cellular membrane, referred to as Descemet's membrane, which is followed by a monolayer of single cell thickness of specialized endothelial cells which forms the posterior surface of the cornea. The endothelial layer does not regenerate and when it is diseased, scratched or otherwise injured, it must be replaced.

In some animal species including human, the corneal endothelium does not normally replicate in vivo to replace cells lost due to injury or aging (Murphy C, et al., Invest. Ophthalmology Vis. Sci. 1984; 25:312-322; Laing R A, et al., Exp. Eye Res. 1976; 22:587-594). However, human corneal cells can be cultured in vitro with a growth factor-enriched, fetal calf serum-containing medium under normal tissue culture conditions (Baum J L, et al., Arch. Ophthalmol. 97:1136-1140, 1979; Engelmann K, et al., Invest. Ophthalmol. Vis. Sci. 29:1656-1662, 1998; Engelmann K, and Friedl P; In Vitro Cell Develop. Biol. 25:1065-1072, 1989). If the cultured cells can be utilized to replace the loss of corneal endothelial cells it will greatly enhance the donor pool of human corneas. This is important as one may be able to augment the donor corneas currently rejected for transplantation procedures due to inadequate endothelial cell counts (Gospodarowicz D, et al., Proc. Natl. Acad. Sci. (USA) 76:464-468, 1979; Gospodarowicz D, et al., Arch. Ophthalmol. 97:2163-2169, 1979). This pool of corneas, rejected due to low endothelial cell density, makes up to 30% of the total donated corneas annually (National Eye Institute: Summary report on the cornea task force. Invest Ophthalmol Vis Sci 12:391-397, 1973). Furthermore, a method to culture human corneal endothelial cells from a low initial density, and the ability to reseed the cells grown in vitro onto denuded corneal buttons, will enable the use of the recipient's own undamaged stroma for allo-cell and auto-stroma type of transplantation (Insler M S, and Lopez J G, Cornea 10:136-148, 1991).

Tissue culture techniques are being successfully used in developing tissue and organ equivalents. The basis for these techniques involve collagen matrix structures, which are capable of being remodeled into functional tissue and organs by employing the right combination of living cells, nutrients, and culturing conditions. Tissue equivalents have been described extensively in many patents, including U.S. Pat. Nos. 4,485,096; 4,485,097; 4,539,716;. 4,546,500; 4,604,346; 4,837,379; and 5,827,641, all of which are incorporated herein by reference. One successful application of the tissue equivalent is the living skin equivalent, which has morphology similar to actual human skin. The living skin equivalent is composed of two layers: the upper portion is made of differentiated and stratified human epidermal keratinocytes that cover a thicker, lower layer of human dermal fibroblasts in a collagen matrix. Bell, et al., “Recipes for Reconstituting Skin,” J. of Biochemical Engineering, 113:113-119 (1991).

Studies have been done on culturing corneal epithelial and endothelial cells. Xie, et al., “A simplified technique for the short-term tissue culture of rabbit corneal cells,” In Vitro Cellular & Developmental Biology, 25:20-22 (1989) and Simmons, et al., “Corneal Epithelial Wound Closure in Tissue Culture: An in vitro Model of Ocular Irritancy,” Toxicology and Applied Pharmacology, 88:13-23 (1987).

Until the advent of the present invention, prior art methods of culturing human corneal endothelial cells (HCEC) encountered problems such as the fact that HCEC cells could only be seeded at high cell density (2000-5000 cells/square mm) therefore limiting the possibility to start a primary culture from small specimen, and that HCEC cells could not be passaged continuously at low seeding density (50-100 cells/square mm) which limits the ability to expand the HCEC stock for storage and future use.

### SUMMARY OF THE INVENTION

The present invention provides for a method of culturing HCEC on a novel extracellular matrix which enables the establishment of primary cultures from small specimens of HCEC (100-500 cells). The present invention also provides for a method which to expand these primary HCEC colonies via serial passage into large quantity of cells for transplantation and cryostorage for future use.

A method for initiating primary cultures of corneal endothelial cells, including that of human origin, by using dissection enables the culture to start from a low initial density (100-500 cells/mm²) and expand from a seeding density of 1 to 32. These cells can be effectively passaged 7 to 8 times without losing their morphological integrity and physiological functions, such as formation of tight intracellular junctions and Na/K pump activation. The corneal endothelial cultures can be maintained in a commonly used fetal bovine serum (FBS) supplemented culture medium enriched with selected growth factors such as fibroblast growth factors 1 and 2 (FGF1, FGF2), epidermal growth factors (EGF), transforming growth factor β (TGFβ), endothelial cell growth factor (ECGF), and other growth factors known in the art of cell culture. In particular, if the corneal endothelial cells are propagated in a natural extracellular matrix as supplied by bovine corneal endothelial culture, or a synthetic attachment protein mixture containing such components as fibronectin, laminin, collagen type I and IV, and RGDS, or on a carbon plasma deposit known as diamond-like carbon (DLC), the culture will assume a more hexagonal morphology upon repeated passage at high split ratio (1:32 or 1:64) for up to 10 passages. The generation of a large pool of corneal endothelial cells, especially that of human origin, can be banked in cryo-storage and used for future cell transplantation procedures.

It is therefore an object of the present invention to provide a method of cell culture of HCEC that can be used for the establishing and serial culturing of other cell types of human origins such as neurons, pancreatic beta cells, and chrondocytes.

It is another object of the present invention to create HCEC in sufficient quantities of HCEC that can be used for other purposes.

It is a further object of the present invention to provide an in vitro cell culture model of the human cornea.

It is also an object of the present invention to provide a means for regenerating corneal endothelial cells in a cornea by replacing the damaged corneal endothelial cells with HCEC grown in culture system of the present invention.

It is yet a further aspect of the present invention to provide a means for regenerating other types of damaged human and mammalian endothelial cells by replacing the damaged endothelial cells with endothelial cells grown in the culture system of the present invention.

These and other objects of the invention, as well as many of the attendant advantages thereof, will become more readily apparent when reference is made to the following detailed description of the preferred embodiments.

### BRIEF DESCRIPTION OF THE FIGURES

FIG. 1 shows generation curves for long term serial propagation of cultured human endothelial cells on different substrates.

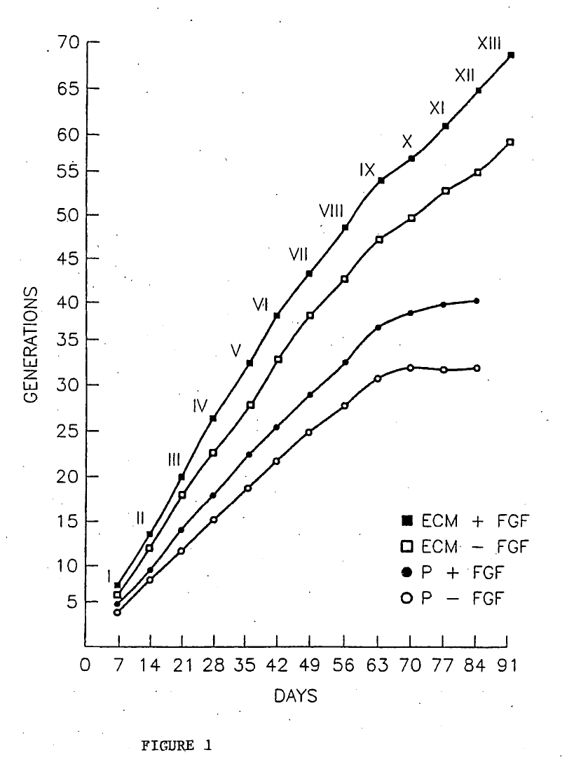

FIG. 2 illustrates the effects of various attachment factors on the proliferation of cultured human corneal endothelial cells in the presence or absence of bFGF.

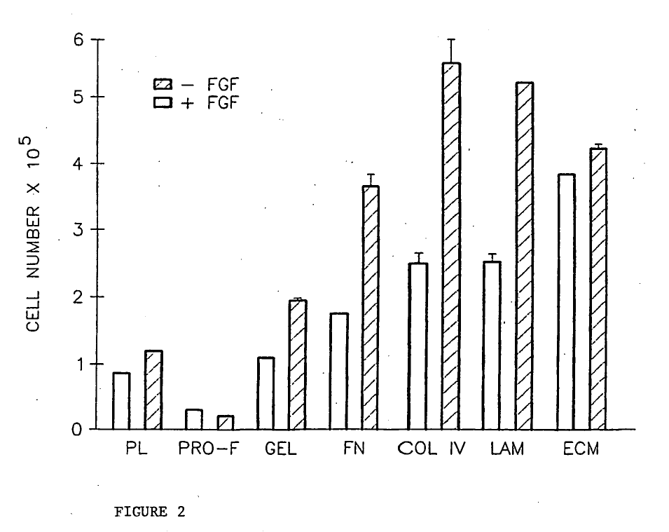

FIG. 3 is a time curve of attachment of cultured human corneal endothelial cells onto the denuded human corneal buttons coated with attachment agents.

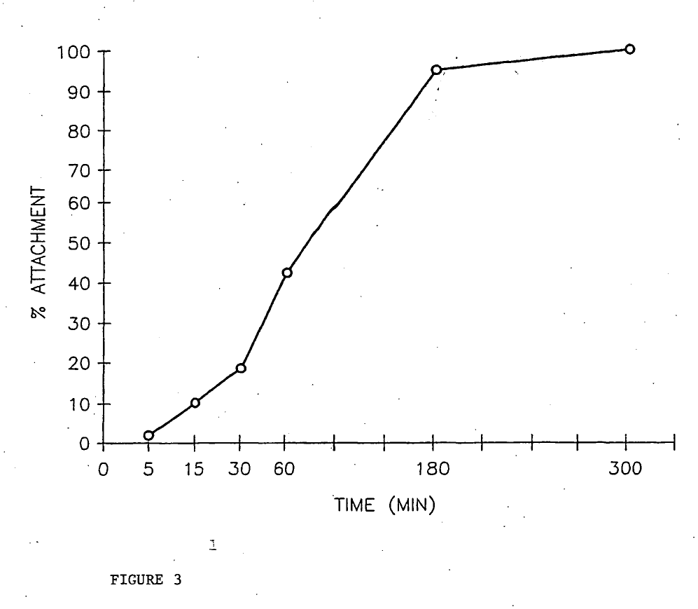

### DETAILED DESCRIPTION AND PREFERRED EMBODIMENTS

In the first step of constructing the in vitro cornea model, the endothelial cells are seeded onto membranes of a cell culture insert. These endothelial cells will form the inner layer, or basal layer, of the corneal equivalent.

We have developed a system where the native corneal endothelium from a donor cornea, whose endothelial cell population is inadequate in cell count or damaged due to disease or physical disruption, can be removed without harming the integrity of the Descemet's membrane and the structure and function of the stromal later. The preparation of such a denuded cornea can be achieved with treatment from a diluted detergent such as Triton X-100 (0.5-5%) or ammonium hydroxide (at concentrations ranging from 10 mM to 200 mM) for a time period ranging from 2 minutes to 60 minutes at a temperature of 4° C. to 25° C. The denuded corneas will be washed extensively 8-10 times with phosphate buffered saline (PBS) to remove any detergent or ammonium hydroxide residue. The denuded corneas will then be ready for cell coating from the cultured human corneal endothelial cell stock.

Prior to seeding the cells onto the denuded corneal surface, a predetermined mixture of attachment proteins containing fibronectin (ranging from 0.1 μg to 500 μg/ml in PBS), laminin (0.1 μg to 500 μg/ml in PBS), RGDS (0.01 μg to 100 μg/ml in PBS), collagen type IV (ranging from 0.1 μg to 1000 μg in 0.1 M acetic acid) will be added to the denuded surface (Descemet's membrane) and incubated at 4° C. for a period ranging from 5 to 60 minutes. The residual protein mixture will be removed after the incubation period, and the cornea is rinsed three times with PBS and placed endothelial side up on a Teflon concave holder. An 11 mm diameter button will be punched out with a size 11 trephine. This button will be ready to receive the cultured corneal endothelial cells.

The cultured human endothelial cells will be removed from the tissue culture dish with 0.05% trypsin and 0.02% EDTA in saline solution. The cell suspension will be counted with a Coulter Particle Counter (Z1 model, Beckman-Coulter) and a preparation of about 50,000 to 500,000 cells/ml, preferably about 200,000 cells in 200 μl of culture medium (DME-H16 with 5% fetal calf serum or a serum-free medium containing a mixture of attachment proteins such as fibronectin, laminin, and fibroblast growth factors (at 10 ng to 400 ng/ml) will be added carefully onto the denuded corneal button. A layer of 1% sodium hyaluronate, such as Healon® (Advanced Medical Optics, Santa Ana, Calif.) at approximately 0.1 to 0.5 ml, will be layered onto the cell suspension as a protectant. The corneal button will then be incubated at 37° C. in a 10% CO₂ incubator for a period of 10 minutes up to 24 hours. Alternatively, the coated corneal button will be incubated for 20 minutes and the cornea will be rinsed three times with PBS at 25° C. and ready for transplantation.

Alternatively, the process of maintaining human corneal endothelial cells in culture, expansion of the corneal endothelial cells, and the preparation of the attachment protein can be used to coat artificial cornea stroma generated from polymer-gel composition. Briefly, a poly-gel stroma can be molded into a cornea shape, and the concave side (endothelial side) will be treated with a mixture of attachment proteins and growth factors such as fibronectin (ranging from 0.1 to 500 μg/ml in PBS), laminin (ranging from 0.1 to 500 μg/ml in PBS), RGDS (ranging from 0.01 to 100 μg/ml in PBS), collagen type IV (ranging from 0.1 μg to 1000 μg in 0.1 M acetic acid), FGF (10 to 400 ng/ml in PBS), EGF (10 to 400 ng/ml in PBS), or TGFβ (1 to 100 ng/ml in PBS). After incubation at 4° C. for a period ranging from 10 minutes to 2 hours, the artificial stroma will be rinsed three times with PBS, and cultured human corneal endothelial cells at a density of about 50,000 to about 10⁶ cells/ml preferably about 150,000 to 250,000 cells/200 ml of culture medium (DMA-H16 with 5% FCS or a mixture of attachment proteins containing fibronectin, laminin, RGDS, and collagen type IV) will be added to a corneal button of 11 mm diameter. A layer of (10 mg/mL sodium hyaluronate, 0.1 to 0.5 ml) will be applied carefully onto the cell layer as a protectant, and the button will be incubated at 37° C. in a 10% CO₂ incubation for a period ranging from 10 minutes to 24 hours. The artificial corneal button will be rinsed 3 times with PBS after the incubation and will be ready for transplantation.

In alternative embodiments, the corneal endothelial cells used to form the endothelial layer can be derived from a variety of mammalian sources. Non-transformed corneal endothelial cells derived from sheep, rabbit, and cows have been used. Mouse corneal endothelial cells have been transformed with large T antigen of SV40. (Muragaki, Y., et al., Eur. J. Biochem. 207 (3):895-902 (1992).) Non-human cell types which can be used also include transformed mouse corneal endothelial cell lines, or normal corneal endothelial cells derived from sheep or rabbit. The normal rabbit endothelial cells can be derived from enzymatically dissociated corneal endothelium or from explants of cornea and are serially cultivated in MSBM medium (Johnson, W. E. et al., In Vitro Cell. Dev. Biol. 28A:429-435 (1992) ) modified by the addition of 50 μg/mL heparin and 0.4 μg/mL heparin binding growth factor-1 (MSBME).

In yet another embodiment, endothelial cells from a non-corneal origin may also be used in this invention. The non-corneal origin endothelial cells that have also been used in this invention include ovine and canine vascular and human umbilical vein endothelial cells. The endothelial cells may be transformed with a recombinant retrovirus containing the large T antigen of SV40 (Muragaki, et al., 1992, supra). Transformed cells continue to grow in the corneal equivalent and form mounds on top of the acellular layer due to their lack of contact inhibition. Non-transformed cells will form a monolayer underlying the stromal cell-collagen layer. Alternatively, normal endothelial cells may be transfected as above, but with the addition of a recombinant construct that expresses a heat sensitive gene. These transformed cells will grow in continuous culture under reduced temperature. After establishment of a confluent endothelial cell layer, the temperature can be raised to deactivate the transforming gene, allowing the cells to resume their normal regulation and exhibit contact inhibition, to form an endothelial cell monolayer similar to the non-transformed cells. Most peptides are heat sensitive (with the exception of heat shock proteins) so that there is a wide choice of peptides that can be deactivated by raising culturing temperature. Transformation in this way also facilitates the use of hard to obtain and cultivate cell types such as human corneal endothelial cells.

Preparation of Extracellular Matrix (ECM) Coated Plates for Primary Corneal Endothelial Culture and Subsequent Passaging.

Bovine corneal endothelial cells (BCEC) in culture will be seeded onto dishes in a DME-H16 medium containing 10% FCS, 5% CS, 5% Dextran, 300 ug/ml glutamine, 2.5 ug/ml Amphotericin B, and 50 ng/ml bFGF. At confluency, (7-10 days post seeding), the dishes will be treated with 20 mM NH₄OH at a volume sufficient to cover at least ⅔ of the plate. After 5 minutes of shaking in a mechanical shaker, the NH₄OH will be aspirated and the dished rinsed 5 times with PBS. The dishes will be stored at 4° C. at least a week prior to use in order to eliminate any surviving BCEC. Laminin and fibronectin will be dissolved in distilled water at a concentration of 100 μg/ml. Type IV collagen will be dissolved in 0.6% v/v acetic acid/water. Laminin, fibronectin, and type IV collagen will be added to the ECM plates as needed for culture purposes.

### EXAMPLE 1

### Non-Enzymatic Dissection of Primary Human Corneal Endothelial Cells

The corneal rims from human donors (after the central portion has been removed for transplantation) or whole donor corneas will be rinsed in a large volume (50 ml) of phosphate buffered saline (PBS). They will then be placed in endothelial side up on a holder. The trabecular meshwork and remnants of iris will be removed carefully by micro-dissection. By using sharp pointed jeweler's forceps, the endothelial cell layers and the Descemet's membrane will be peeled off very carefully with great care taken not to include any underlying stromal tissue. This step can be confirmed by viewing the dissected Descemet's membrane under an inverted microscope to make sure it only carries the corneal endothelial cells on one side and nothing on the other side. The piece of tissue will be placed onto an ECM coated 35 mm tissue culture dish or similarly suitable container, filled with approximately 0.5 ml of culture medium (DME-H16 with 15% fetal calf serum enriched with b-FGF at 250 ng/ml). The dish will be incubated at 37° C. in a 10% CO₂ incubator for 24 hours, and then another 1 ml of culture medium will be added. The sample will be incubated without disturbance for about 7 days to see if a colony of corneal endothelial cells migrates outwards from the tissue sample, at which time (7 to 14 days after the sample is placed in culture) the medium is changed every other day until the cell count reaches about 200-500 cells.

### EXAMPLE 2

### Culture of Human Corneal Endothelial Cells at High Split Ratio

When the primary cell count from the tissue sample outgrowth reaches a number of between 150 to 750, preferably between about 200 to 500, the cells will be released from the dish with STV solution (0.05% trypsin, 0.02% EDTA in normal saline). The STV solution will be removed when the cells round up but are still attached to the culture dish. No centrifugation step is necessary since the remaining STV will be inactivated by the growth media containing 15% fetal calf serum. The corneal cells will be placed onto a 60-mm ECM-coated dish (between about 250 to 1000 cells, preferably about 500 cells per dish). The medium will be changed every other day and b-FGF at a concentration of 250 ng/ml will be added at the time of medium change. At confluence (about 7 to 10 days after plating), the cells will be passaged again at the same split ratio (1:16 to 1:64) or will be frozen in 10% DMSO, 15% FCS at a density of between about 5×10⁵ to about 5×10⁶ cells/ml, preferably about 10⁶ cells/ml per ampoule and stored in liquid nitrogen for future use. The passaging can be carried out for up to 8 times without loss of cell functions or morphological integrity.

Freezing of HCEC Stock.

For each of the 5 ml of HCEC collected, 0.5 ml of DMSO was added to the cell suspension. Each 1.1 ml of the mixture was aliquoted into a 1.5 ml cryopreservation tube to yield an approximate 1 million cells per vial final concentration. The vials were then put into a Styrofoam box and let stand in a −80° C. freezer for 24 hours. After 1 day, the ampoules were transfer into liquid nitrogen for long term storage.

### EXAMPLE 3

### Denudation of Corneal Button

Human donor corneal buttons are obtained from the Eye Bank. These corneal buttons are deemed unsuitable for transplantation due to inadequate endothelial cell counts, but otherwise are healthy and disease free and obtained under eye banking guidelines.

The corneal button will be placed endothelial side up in a holder, and rinsed three times with PBS. Then a solution of ammonium hydroxide at a concentration ranging from 10 mM to 200 mM will be added carefully into the corneal button without spilling over the top. The cornea will be kept at temperatures of about 10° C. to 25° C. for a period of 5 minutes up to 2 hours. Then the ammonium hydroxide will be removed, and the inside of the cornea button rinsed approximately 10 times with PBS. A cotton swab will be slid gently across the endothelial surface to remove any residual cell skeletons or debris. The corneal button is rinsed again three times with PBS, punched with an 11 mm trephine, and is then ready for coating with cultured human corneal endothelial cells.

Alternatively, the native corneal endothelium can be removed by adding Triton-X100 at a concentration of 0.5 to 5% in distilled water kept at 10° C. for a period ranging from 5 minutes to 2 hours, and then processed as previously described. Furthermore, the corneal endothelium can be treated with distilled water for a period of 20 minutes to 2 hours at a temperature ranging from 4° C. to 25° C. Then the cotton swab will be slid gently across the endothelial surface to remove the cell cytoskeleton and debris. The cornea will then be processed with an 11 mm trephination.

### EXAMPLE 4

### Treatment of Denuded Corneas with Attachment Proteins and Growth Factors

After trephination, the denuded cornea button will be placed endothelial side up again in a holder. A solution of attachment proteins containing fibronectin at a concentration ranging from 10 μg to 500 μg/ml in PBS, laminin (10 82 g to 500 μg/ml in PBS), RGDS (1 μg to 100 μg/ml in PBS), collagen type IV (10 μg to 1000 μg in 0.1 M acetic acid), b-FGF (1 to 500 ng/ml in PBS), EGF (1 ng to 500 ng/ml in PBS) will be added carefully onto the denuded cornea button. The specimen is allowed to incubate at 4° C. for a time ranging from 5 minutes to 2 hours, at the end of which the cocktail will be removed and the cornea rinsed 3 times with PBS.

### EXAMPLE 5

### Coating of Denuded Cornea with Cultured Human Endothelial Cells

The cultured cornea endothelial cells will be removed from the culture dish with STV solution (0.05% trypsin, 0.02% EDTA in saline) as previously described. The cells will be centrifuged at 2000 rpm for 5 minutes and the culture medium removed. The cell pellet will be resuspended with 2 ml of DME-H16 medium containing fetal calf serum at a concentration of 0.1 to 5%. About 100 μl of the suspension will be counted in a Coulter Particle Counter to determine the cell number per ml. Then the cell concentration will be adjusted to about 5×10⁵ to 10⁷ cells/ml, preferably about 10⁶ cells/ml and 200 μg of the cell suspension will be added carefully added into the 11 mm trephined corneal button. A layer of sodium hyaluronate, at a concentration of about 10 mg/ml, ranging from 0.2 to 0.5 ml (Healon®) will be overlaid carefully onto the cell suspension as a protectant. The corneal button will then be incubated at 37° C. in a 10% CO₂ incubator for a period of 20 minutes to 24 hours. The human corneal endothelial cell coated button will be ready for transplantation.

In an alternate embodiment, an artificial matrix can be generated although such material may not be as effective in promoting the growth and morphological integrity (hexagonal shape) of the HCEC.

To generate the artificial matrix, fibronectin, laminin and RGDS will be dissolved at concentrations of about 100 82 g/ml in distilled water, and collagen type IV is dissolved at a concentration of about 1 mg/ml in 0.01% acetic acid. Basic FGF is dissolved at a concentration of about 100 μg/ml in bovine serum albumin (0.05% w/v). All the materials are mixed together in a 15 ml centrifuge tube and swirled gently to avoid bubbling. The mixture is then incubated at 4° C. for two hours.

To coat the tissue culture dishes, the mixture is diluted 1:10 with phosphate buffered saline, and then 1 ml of the solution is added to a 35 mm dish and store at 4° C. for 1 hour. Prior to use the solution is aspirated and the cell suspension will be added to the dish.

Having described the invention, many modifications thereto will become apparent to those skilled in the art to which it pertains without deviation from the spirit of the invention as defined by the scope of the appended claims.

The disclosures of U.S. patents, patent applications, and all other references cited above are all hereby incorporated by reference into this specification as if fully set forth in its entirety.

1. The method of non-enzymatic harvesting and in vitro culturing corneal epithelial cells for transplantation comprising the steps of: a) dissecting corneal endothelial cells from a tissue source; growing said corneal endothelial cells at a low density in extracellular matrix coated (ECM) culture plates for a period of time; b) passaging said cells into a secondary culture system wherein the secondary culture system is comprised of ECM coated culture plates and the addition of sufficient cellular growth factors; c) growing the corneal endothelial cells to confluency; and d) harvesting the corneal endothelial cells from the second culture system in sufficient quantities to be useful in transplantation to a subject in vivo.

2. The method of claim 1 wherein the dissection of the corneal endothelial cells further comprises the step of removing the cells from the tissue source such that the cells are dissected away from the stroma of said tissue source prior to growing in the first culture system.

3. The method of claim 2 wherein said tissue source can be corneal buttons or rims.

4. The method of claim 2 wherein the ECM is comprised of Bovine corneal endothelial cell extracellular matrix (BCE-ECM).

5. The method of claim 2 wherein the ECM is comprised of an artificially generated extracellular matrix (AG-ECM).

6. The method of making ECM coated plates comprising the steps of: a) seeding bovine corneal endothelial cells (BCEC) onto dishes in DME-H16 medium containing approximately 10% Fetal Calf Serum, 5% Calf Serum, 5% Dextran, 300 μg/ml glutamine, 2.5 μg/ml Amphotericin B, and 50 ng/ml bFGF; b) growing BCEC until confluent; c) treating the dishes will be treated with NH₄OH at a volume sufficient to cover at least ⅔ of the plate for at least about 5 minutes whereafter the NH₄OH removed; and d) storing the BCEC coated at 4° C. about a week prior to use in order to eliminate any surviving BCEC.

7. The method of claim 5 wherein the artificially generated endothelial cell extracellular matrix (AG-ECM) is made by the process comprising: a) fibronectin, laminin and RGDS (Arg-Gly-Asp-Ser peptide) are prepared in a 100 μg/mL in distilled water; b) collagen type IV is prepared at a concentration of about 1 mg/mL in 0.01% acetic acid ; c) basic fibroblast growth factor (bFGF) is prepared at a concentration of about 100 μg/mL in bovine serum albumin (0.05% w/v); d) the solutions of steps a, b, and c are mixed and then incubated at 4° C. for two hours; and e) the mixture of step d is diluted about 1:10 with phosphate buffered saline, and then a sufficient amount of the solution is added to a dish and allowed to stand at 4° C. for approximately 1 hour before use.

8. Human corneal endothelial cells (HCEC) suitable for use in transplantation made using the method of claim 1.

9. Human corneal endothelial cells (HCEC) suitable for use in transplantation made using the method of claim 2.

10. Human corneal endothelial cells (HCEC) suitable for use in transplantation made using the method of claim 5.

11. An apparatus for growing cells in culture having at least one surface which is in contact with the cells and wherein the surface is coated with a mixture comprising BCE-ECM prior to use.

12. The apparatus of claim 11 selected from the group consisting of: cell culture plates and flasks.

13. The apparatus of claim 11 wherein the cells are mammalian cells.

14. An apparatus for growing cells in culture having at least one surface which is in contact with the cells and wherein the surface is coated with a mixture comprising AG-ECM prior to use.

15. The apparatus of claim 14 wherein the cells are mammalian cells.

16. A method of making HCEC cells wherein said HCEC are lacking class I HLA antigens comprising the steps of: a) dissecting human corneal epithelial cells from a neonatal source such that said cells do not express class I HLA antigens; b) growing said corneal epithelial cells at a low density in a range of about 100 to 500 cells per square millimeter in a primary culture system comprising extracellular matrix coated (ECM) culture plates for a period of time; c) passaging said cells into a secondary culture system wherein the secondary culture system is comprised of an ECM coated culture plates and the addition of sufficient cellular growth factors; d) growing said cells until the cells are confluent; and e) harvesting said cells from the second culture system in sufficient quantities to be useful in transplantation to a subject in vivo.

17. The method of claim 16 wherein the ECM is comprised of Bovine corneal endothelial cell extracellular matrix (BCE-ECM).

18. The method of claim 16 wherein the ECM is comprised of an artificially generated extracellular matrix (AG-ECM).

19. Human corneal endothelial cells (HCEC) suitable for use in transplantation made using the method of claim 16.

20. A method of making HCEC cells wherein said HCEC are lacking class I HLA antigens comprising the steps of: a) dissecting human corneal epithelial cells from a tissue source, growing said corneal epithelial cells at a low density in a range of about 100 to 500 cells per square millimeter in a primary culture system comprising extracellular matrix coated (ECM) culture plates for a period of time; b) passaging said cells into a secondary culture system wherein the secondary culture system is comprised of an ECM coated culture plates and the addition of sufficient cellular growth factors; c) growing said cells to confluency; d) harvesting said cells from the second culture system in sufficient quantities to be useful in transplantation to a subject in vivo; and e) transforming said cells such that the a cell line is created and said cells contain a targeted disruption in the HLA gene locus thereby inhibiting expression of HLA antigens.

21. Human corneal endothelial cells (HCEC) suitable for use in transplantation made using the method of claim 20.

22. The method of claim 1, wherein the genotype of each HCEC cell line is determined using gel-based detection methods, using non-gel-based detection methods or with genetic markers.

23. The method of claim 1, wherein the target immunotype of each HCEC cell line is determined using serological or molecular methods.

24. The method of claim 20, wherein the target immunotype is determined by HLA tissue typing.

25. A cell depository comprising multiple populations of HLA-typed HCEC cell lines, wherein each HCEC cell line is derived from a different donor and is homozygous for a unique HLA haplotype.

26. The cell depository of claim 25, wherein the HCEC cell lines are obtained from donors of different ethnicities.

27. The cell depository of claim 25, wherein the contents of the depository are catalogued.

28. A method for producing an HCEC cell depository of genotyped HCEC cells from multiple donors comprising the steps of: (a) selecting donors; (b) determining the genotype of each donor; (c) isolating HCEC cells from primary cultures obtained from each donor; (d) culturing the isolated HCEC cells to obtain HCEC cell lines; (e) determining the genotype of each HCEC cell line; and (f) cataloging the genotype of each HCEC cell line obtained in (g).

29. A method for producing a HCEC cell depository of immunotyped HCEC cells from multiple donors comprising the steps of: (a) selecting donors; (b) determining the immunotype of each donor; (c) developing primary cultures of HCEC cells; (d) isolating HCEC cells from each donor; (e) culturing the isolated HCEC cells to obtain HCEC cell lines; (f) determining the immunotype of each HCEC cell line; and (g) cataloging the immunotype of each HCEC cell line obtained in (e).

30. A method for producing a HCEC cell depository of genotyped and immunotyped HCEC cells from multiple donors comprising the steps of: (a) selecting donors; (b) determining the genotype and immunotype of each donor; (c) developing primary cultures of HCEC cells; (d) isolating HCEC cells from each donor; (e) culturing the HCEC cells to obtain HCEC cell lines; (f) determining the genotype and immunotype of each HCEC cell line; and (g) cataloging the genotype and immunotype of each HCEC cell line obtained in (e).

31. The method of claim 28, wherein the donors are mammalian.

32. The method of claim 28, wherein the donors are human.

33. The method of claim 29, wherein the donors are human.

34. A method of transporting HCEC for transplantation comprising the steps of: a) growing HCEC according to the method of claim 2 upon a biodegradable polymer membrane to confluency; b) placing the membrane coated with the HCEC into a flask or suitable container filled with culture medium; and c) transporting said membrane coated with the HCEC.

35. The method of claim 34 wherein said target tissue is a corneal button.

36. The method of claim 34 wherein said target tissue is a secondary culture system.

37. A method of transporting HCEC for transplantation comprising the steps of: a) growing HCEC according to the method of claim 2 upon a biodegradable polymer membrane to confluency; b) placing the membrane coated with the HCEC onto a donor target tissue; c) growing the HCEC on the donor tissue for a time period sufficient to prevent dislodging during transportation; and d) transporting said tissue in a storage medium.

38. The method of claim 37 wherein the target tissue is a denuded cornea.

39. The method of claim 34 wherein the biodegradable polymer comprises a semi-solid state suitable for coating with BCE-ECM or other biocompatible coating such as Diamond-Like-Carbon.

40. A method for protecting the regenerated corneal button from denuding during transport or implantation comprising the steps of: a) growing HCEC according to the method of claim 2 upon a biodegradable polymer membrane to confluency; b) placing the membrane coated with the HCEC onto a donor target tissue; c) growing the HCEC on the donor tissue for a time period sufficient to prevent dislodging during transportation in the presence of 1% sodium hyaluronate which has been conjugated to bFGF; and d) transporting said tissue in a storage medium having 1% sodium hyaluronate conjugated to bFGF.

41. The method of claim 40 wherein the target tissue is a denuded cornea.

42. The target tissue treated according to claim 40.

43. A method of storing HCEC regenerated target tissue for transplantation comprising the steps of: a) growing HCEC according to the method of claim 2 upon a biodegradable polymer membrane to confluency; b) placing the membrane coated with the HCEC onto a donor target tissue; c) growing the HCEC on the donor tissue for a time period sufficient to prevent dislodging during transportation; d) adding an anti-icing or cryoprotective agent to said tissue in a storage medium; and e) storage of the target tissue at a very low temperature.

44. A method for denuding a native cornea to make it suitable for transplantation or correction comprising the steps of: a) placing a corneal button or other target tissue in a suitable holder; b) adding a sufficient quantity of denuding reagent to the holder in step a so that it completely covers the target tissue; c) incubating the tissue with the denuding reagent for a sufficient period of time at approximately room temperature; and d) washing the target tissue with an appropriate buffer approximately 10 times.

45. The method of claim 44 wherein the denuding reagent is comprised of a solution of Triton X at a concentration of about 0.01 to 1% v/v in phosphate buffered saline.

46. The method of claim 44 wherein the incubation time is about 5 minutes.

47. The method of claim 44 wherein the denuding reagent is comprised of a solution of ammonium hydroxide at a concentration of about 20 mM.

48. The method of claim 47 wherein the incubation time is between about 2 to 5 minutes.

49. A method for denuding a native cornea to make it suitable for transplantation or correction comprising the steps of: a) placing a corneal button or other target tissue, in a suitable holder; b) adding a sufficient quantity of distilled water to the holder in step a so that it completely covers the target tissue; c) incubating the tissue with the denuding reagent for about 15 minutes at approximately room temperature; d) aspirating off about half the volume of water; e) sweeping the wetted endothelium mechanically from the corneal button; and f) washing the corneal button approximately 3 times with phosphate buffered saline.

50. A reconstituted extracellular matrix preparation comprising: a sufficient amount of growth factor mixture and a sufficient amount of adhesion factor mixture.

51. The growth factor mixture of claim 50 comprising a sufficient quantity of bFGF, EGF and polycarbophyll in a suitable biological buffer.

52. The growth factor mixture of claim 51 wherein the concentrations of bFGF, EGF and polycarbophyll are approximately 3.33 pg/mL, 33.33 pg/mL and 0.33 mg/mL respectively.

53. The adhesion factor mixture of claim 50 comprising a sufficient quantity of laminin, fibronectin, RGDS, and collagen IV in a suitable biological buffer.

54. The adhesion factor mixture of claim 53 wherein the concentrations of laminin, fibronectin, RGDS, are approximately 83.33 μg/mL, and collagen IV is approximately 250 pg/mL.

55. A method of coating a denuded cornea comprising the steps of: a) placing a corneal button or other target tissue in a suitable holder; b) washing the corneal button with phosphate buffered saline; c) adding a sufficient quantity of reconstituted extracellular matrix preparation of claim 50 to the holder in step a so that it completely covers the target tissue; d) incubating the corneal button for a sufficient period of time at approximately 4° C.; and e) washing the corneal button with phosphate buffered saline or other suitable buffer.

56. The method of coating a denuded cornea according to claim 55, further comprising the step of: f) adding approximately 300 μL of 1% sodium hyaluronate to the corneal button prior to seeding of new endothelial cells.
